(19) United States
(12) Patent Application Publication
(10) Pub. No.: US 2017/0340403 A1
(43) Pub. Date: Nov. 30, 2017

(54) ALLERGY TREATMENT KIT

(71) Applicant: Sally Ann Roberts-Monteleone, Harriman, NY (US)

(72) Inventor: Sally Ann Roberts-Monteleone, Harriman, NY (US)

(21) Appl. No.: 15/163,643

(22) Filed: May 24, 2016

(51) Int. Cl. A61B 50/31; A61K 31/137; A61K 31/426; A61B 50/30

(52) U.S. Cl. CPC A61B 50/31; A61K 31/426; A61K 31/137; A61B 2050/314; A61B 2050/3008

(57) ABSTRACT

A portable emergency treatment kit for allergic and anaphylactic reactions. The kit comprises a carry case comprising a first panel and a second panel detachably connected together by a fastening member such as a zipper unit. A plurality of compartments on the inner face of the panels housing at least one oropharyngeal airway device, epinephrine injection, histamine H2 receptor antagonist and allergy relief product. The kit provides ready access to vital medications and airway device for emergency treatment of allergy.

### CROSS-REFERENCE TO RELATED APPLICATIONS

This application claims the priority and benefit of U.S. provisional application No. 62/182,763 titled “Allergy kit”, filed on Jun. 22, 2015. The contents of provisional application No. 62/182,763 is incorporated herein by reference in its entirety for all purposes.

### BACKGROUND OF THE INVENTION

### 1. Field of the invention

The present disclosure relates generally to emergency treatment kits. More specifically, the disclosure relates to a portable emergency kit comprising vital medications and airway tools for the treatment of severe allergic or anaphylactic reactions.

### 2. Description of Related Art

Emergency kits generally contain medications and related items for providing first aid to injured subjects before rushing them to hospital or emergency medical services. The emergency kit comprises minimal set of medical items for providing basic treatment to injured subjects or accident victims. In addition to hospitals and health care centers, it is required to place adequate number of emergency kits in other places such as public transport systems, schools, shopping centers, offices, homes, private vehicles, recreation halls and sports arena.

Existing emergency kits primarily contains medications, bandages and dressing equipment for treating wound injuries, fire related injuries. Emergency kits comprising vital medication combinations are not available for treating severe allergic/anaphylactic reactions. Generally, allergic and anaphylactic reactions caused by food, chemicals or environmental factors. There exists different treatment options for allergic reactions but nothing that provides all aspects of treatment together and none that provide for airway intervention prior to emergency medical services (EMS) or hospital arrival.

Hence, there exists a need for an emergency allergy treatment kit, which is portable and able to provide ready access to vital medications and airway devices for treating allergic and anaphylactic reactions.

### SUMMARY OF THE INVENTION

The present invention relates to a portable emergency treatment kit for allergic and anaphylactic reactions caused by food, chemicals or environmental factors. The kit comprises a carry case comprising a first panel and a second panel detachably connected together by a fastening member such as a zipper unit. A plurality of compartments on the inner face of the panels housing at least one oropharyngeal airway device, epinephrine injection, histamine H2 receptor antagonist and allergy relief product. The kit provides ready access to vital medications and airway device for emergency treatment of allergy. The kit comprises different medications and airway devices for allergy treatment put together in a convenient and easy to access carrying case with premeasured medications according to weight/age.

In an embodiment, the kit comprises a carrying case with multiple pouches containing at least one oropharyngeal airway device, epinephrine injection, sublingual diphenhydramine and histamine H2 receptor antagonist such as famotidine. The emergency treatment kit aids in temporarily managing allergic reactions prior to obtaining proper medical intervention. The kit adds different allergy treatments together in order to make them accessible in case of an emergency related to allergy or severe anaphylaxis. The kits can be customized with varying medication dosages and airway device sizes suitable for children and adults of different age groups.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DIFFERENT VIEWS OF DRAWINGS

FIG. 1 is a perspective view of the emergency kit in an unpacked state showing components of the kit.

FIG. 2 is a rear perspective view of the emergency kit in a packed state.

### DETAILED DESCRIPTION OF DIFFERENT EMBODIMENTS

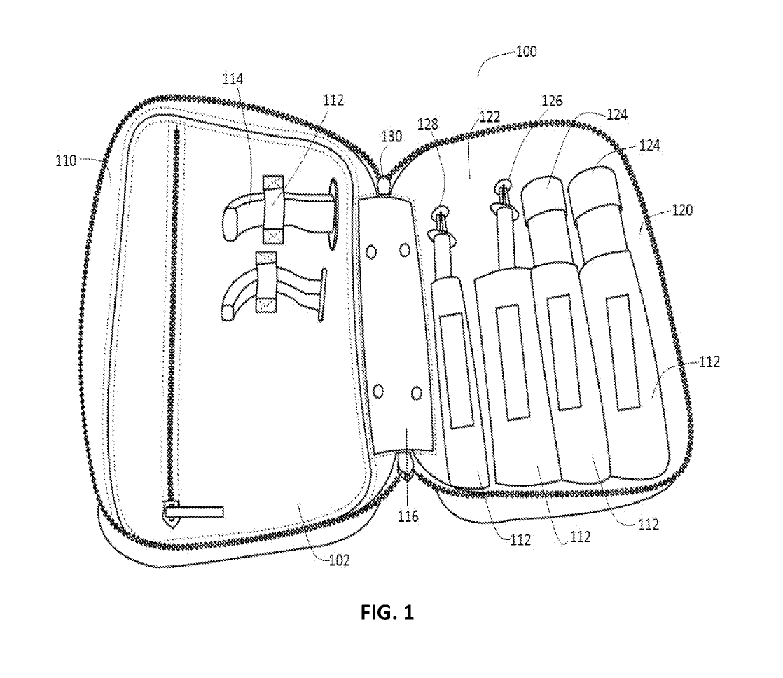

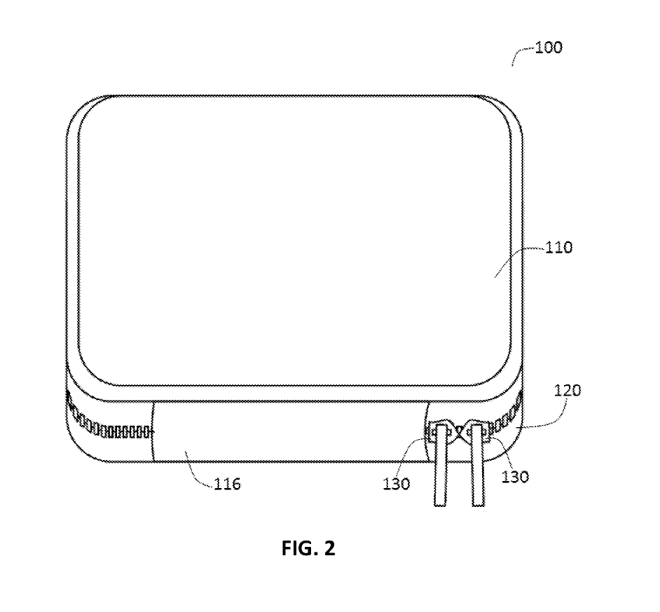

Referring to FIG. 1, showing a perspective view of a portable emergency treatment kit. The emergency kit 100 is used for treating allergic and anaphylactic reactions caused by food, chemicals or environmental factors. The kit 100 is a carry case comprising a first panel 110 and a second panel 120 detachably connected together by a fastening member 130. The fastening member 130 may comprise zipper unit, hook and loop fastener, snap fastener, and other common types of fasteners that allow unfastening the carry case to access its contents and fasten the carry case panels 110, 120 after use. FIG. 2 shows a top perspective view of the emergency kit 100 in a packed state. A portion of the first panel 110 and second panel 120 are joined together through a flexible connecting member 116. In an embodiment, the carry case comprises two fastening members 130.

The kit 100 comprises a plurality of compartments 112 on the inner faces 102, 122 of the respective panels 110, 120. The compartments 112 comprises pockets for housing preferably at least one oropharyngeal airway device 114, epinephrine injection 124, histamine H2 receptor antagonist 126 and allergy relief product 128. The panels 110, 120 may comprise additional pouches for storing prescriptions, instruction manuals, first aid information and emergency contact details. The emergency treatment kit 100 of the present invention is for treating allergic and anaphylactic reactions caused by food, chemicals or environmental factors. The panels 110, 120 are connected by the flexible connecting member 116, which acts as a hinge portion while opening and closing the carry case.

Stimulation of H2 receptor by massive histamine release during anaphylaxis is responsible for many of the hemodynamic complications such as arrhythmia and decreased blood pressure, which may lead to fatal consequences in some cases. Therefore, the kit 100 comprises a medicinal preparation containing histamine H2 receptor antagonist. In an embodiment, the histamine H2 receptor antagonist 126 comprises famotidine, sold under the tradename PEPCID.

Histamine is a natural substance released within the body during times of stress and allergy. Histamine is a biologically active amine produced as a part of local immune response to cause inflammation. In an embodiment, the allergy relief product 128 comprises an antihistamine compound. Antihistamine compounds inhibits the physiological effects of histamine In an embodiment, the antihistamine compound comprises diphenhydramine, sold under the tradename BENADRYL. In an embodiment, the epinephrine injection 124 comprises an auto-injector device filled with measured doses of epinephrine, sold under the tradename EPI-PEN.

The carry case of the kit 100 comprises all medical items required to provide initial emergency treatment for allergy. The allergy relief and histamine H2 receptor antagonist medicinal preparations 128, 126 are formulated and stored in single dose packaging, which enables rapid administration of medication in the event of allergy. Oral airways 114 of different sizes are stored in the carry case for deployment in the event of airway obstruction. The kit 100 provides ready access to vital medications and airway device 114 for emergency treatment of allergy. The kit 100 comprises different medications and airway devices 114 for allergy treatment put together in a convenient and easy to access carrying case with premeasured medications according to weight/age.

In an exemplary embodiment, upon onset of an allergic reaction, a user carrying the emergency kit 100 would be able to administer epinephrine injection 124 followed by allergy relief medication and histamine H2 receptor antagonist 128, 126 to act in conjunction with epinephrine 124. In the event of severe mouth and tongue swelling, an appropriate sized oral airway device 114 can be inserted into the subject's oral cavity and positioned to facilitate air exchange while waiting for appropriate medical intervention.

The emergency treatment kit 100 aids in temporarily managing allergic reactions prior to obtaining proper medical intervention. The kit 100 adds different allergy treatments together in order to make them accessible in case of an emergency related to allergy or severe anaphylaxis. The kit 100 can be customized with varying medication dosages and airway device sizes suitable for children and adults of different age groups.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. An emergency treatment kit for allergy, the kit comprising:
A carry case comprising a first panel and a second panel detachably connected together by a fastening member; and
A plurality of compartments on inner face of the first panel and the second panel housing at least one oropharyngeal airway device, at least one epinephrine injection, at least one histamine H2 receptor antagonist and at least one allergy relief product.

2. The emergency treatment kit of claim 1, wherein the fastening member comprise a zipper fastener.

3. The emergency treatment kit of claim 1, wherein the histamine H2 receptor antagonist comprises a famotidine.

4. The emergency treatment kit of claim 1, wherein the allergy relief product comprises an antihistamine.

5. The emergency treatment kit of claim 4, wherein the antihistamine comprises diphenhydramine.

6. The emergency treatment kit of claim 1, wherein the epinephrine injection comprises an auto-injector device filled with measured doses of epinephrine.

7. The emergency treatment kit of claim 1 is portable.
